(19) United States
(12) Patent Application Publication
(10) Pub. No.: US 2017/0183215 A1
(43) Pub. Date: Jun. 29, 2017

(54) Connected Fuel System

(71) Applicant: Central Illinois Manufacturing Company, Berment, IL (US)

(72) Inventors: Jeffrey Alan Ayers, Decatur, IL (US); Matthew David Valentine, Bement, IL (US); Eric Briner Packard, Champaign, IL (US)

(21) Appl. No.: 15/392,376

(22) Filed: Dec. 28, 2016

Related U.S. Application Data
(60) Provisional application No. 62/271,805, filed on Dec. 28, 2015.

(51) Int. Cl. B67D 7/32; B67D 7/08; G01N 33/28

(52) U.S. Cl. CPC B67D 7/3281; G01N 33/28; B67D 7/08; B67D 7/32; F02D 2200/0611

(57) ABSTRACT

Disclosed herein is a system and method for reporting fuel quality or fuel equipment status, more specifically, systems and methods for detecting and reporting quality, contaminates, cleanliness, and/or free or emulsified water in fuel, as well as the status of a fuel dispenser or dispensing fuel filter. The fuel quality or fuel equipment status may be reported in real time, or near real time, to a remote device or system for further analysis.

### CROSS-REFERENCE TO RELATED APPLICATION

The present application claims the benefit under 35 U.S.C. §119(e) of U.S. Provisional Patent Application Ser. No. 62/271,805 titled “Fuel Evaluation And Monitoring System,” filed Dec. 28, 2015, the contents of which are hereby incorporated by reference.

### TECHNICAL FIELD

The present invention relates to a system and method for reporting fuel quality or fuel equipment status, more specifically, systems and methods for detecting and reporting quality, contaminates, cleanliness, and/or free or emulsified water in fuel, as well as the status of a fuel dispenser or dispensing fuel filter. The fuel quality or fuel equipment status may be reported in real time, or near real time, to a remote device or system for further analysis.

### BACKGROUND

Solid particle contamination and fuel cleanliness is concern in view of efficiency and emissions requirements. Fuel is typically delivered to ASTM standards, which do not specify an ISO 4406 cleanliness code. Vehicle manufacturers, however, specify the permissible fuel cleanliness codes. For example, contamination can plug carburetor jets (or injection nozzles) and otherwise interfere with the operation of an internal combustion engine. Further, certain vehicle manufacturers have written permissible cleanliness codes into their warranty statements. Therefore, fuel is typically filtered by a dispensing fuel filter at the time it is dispensed at, for example, a service station, or storage container. For example, when fuel is transferred from a fuel storage container to a vehicle's fuel tank via a fuel transfer pump, a dispensing fuel filter may be used to remove harmful particles from the fuel. Example dispensing fuel filters include those by Cim-Tek® Filtration, which are available from Central Illinois Manufacturing Company of Bement, Ill. The fuel is typically filtered again at its point of use by a second fuel filter (e.g., a filter coupled with an internal combustion engine).

Selecting the correct dispensing fuel filter can by onerous. The worldwide fuel charter currently calls for an ISO 18/16/13 fuel, but fuel may not be sufficiently clean for modern high-pressure common rail fuel injected diesel engines. Selecting a suitable dispensing fuel filter presently requires taking one or more fuel samples and sending the samples to a lab for ISO 4406 evaluation. In addition to being costly, by the time the lab processes the samples and generates a report for the operator, the fuel has cycled through the system. As a result, operators must rely on outdated information and are left to effectively guess as to what dispensing fuel filter is needed to protect a given piece of equipment. As a result, many operators will select a dispensing fuel filter that exceeds the requirements, thereby increasing the cost to the operator.

In addition to particle contamination and fuel cleanliness, another troubling fuel contaminant is water, especially in alcohol-blended fuels. Alcohols are often added to fuel to, inter alia, boost octane, oxygenate, extend fuel supply, replace ethers, and reduce the impact of fossil fuels on the carbon cycle. Alcohol-blended fuels, however, react differently in the presence of water than alcohol-free fuels. That is, with alcohol-free fuels, water is heavier than the fuel and simply drops to the bottom of the fuel tank. Thus, as long as a proper maintenance protocol is followed, the water level in the fuel tank should not reach the level of an intake for a pump that draws the fuel from the fuel tank. Unlike alcohol-free fuels, however, alcohol-blended fuels separate into two or more layers when exposed to excess water. The two or more layers typically include a denser, alcohol-water layer, and a less dense, fuel layer that is depleted in octane rating and alcohol soluble hydrocarbons. This separation is more commonly known as phase separation, or a phase separation condition. For example, ethanol-blended fuels (a common type of alcohol-blended fuel) contain ethanol, which is hygroscopic, meaning that it seeks out, and retains, water. At low water level concentrations, the ethanol is able to retain the water it has dissolved and remain associated with the fuel. That is, the fuel, water, and alcohol mixture remains stable and usable as a motor fuel. Once the water concentration exceeds a temperature-dependent threshold (e.g., the saturation point) for a given alcohol concentration, fuel-hydrocarbon content, and additives in the fuel (which typically contain alcohol as a major component), the ethanol and water phase separates from the fuel mixture. Under average temperature conditions in the United States, for example, water content of 0.3% to 0.5% by volume is typically a range within which phase separation begins to occur. The alcohol-water layer does not support combustion in a conventional gasoline engine, such as those in vehicles and generators, and, if introduced to the engine, may result in malfunction of internal combustion (e.g., engine stalling). Water may also damage expensive engine components, particularly fuel injectors. Further, the cleanliness of fuels, primarily diesel, has come under increased scrutiny.

In view of the foregoing, a need exist for an improved system and method for reporting fuel quality or fuel equipment status, more specifically, systems and methods for detecting and reporting quality, contaminates, cleanliness, and/or free or emulsified water in fuel, as well as the status of a fuel dispenser or dispensing fuel filter. The fuel quality or fuel equipment status may be reported in real time, or near real time, to a portable user device (e.g., a portable computer, tablet, smart phone, or other device) and/or a remote fuel evaluation and monitoring server for further analysis.

### SUMMARY OF THE INVENTION

The present invention relates to a system and method for reporting fuel quality or fuel equipment status, more specifically, systems and methods for detecting and reporting quality, contaminates, cleanliness, and/or free or emulsified water in fuel, as well as the status of a fuel dispenser or dispensing fuel filter. The fuel quality or fuel equipment status may be dynamically reported (e.g., reported in real time or near real time) to a remote device or system for further analysis.

According to a first aspect, a fuel monitoring system for use with a fuel dispenser comprises: at least one sensor to dynamically monitor a parameter of said fuel dispenser or a volume of fuel passed by said fuel dispenser; a processor operably coupled with said at least one sensor, the processor being configured to receive measurement data from said at least one sensor that represents a monitored parameter of said volume of fuel; and a wireless transceiver operably coupled with said processor that is configured to wirelessly communicate said measurement data from said fuel monitoring system to a portable user device.

In certain aspects, the measurement data is wirelessly communicated to said portable user device as unprocessed measurement data.

In certain aspects, the unprocessed measurement data is processed by said portable user device to determine whether an alert condition at the fuel dispenser is established.

In certain aspects, the measurement data is wirelessly communicated to said portable user device using Bluetooth, infrared, or Wi-Fi.

In certain aspects, the measurement data is communicated to said portable user device via the Internet.

In certain aspects, the fuel monitoring system further comprises a fuel cutoff device to disable flow of fuel from the fuel dispenser. The fuel cutoff device may be configured to disable flow of fuel from the fuel dispenser when, based on said measurement data, an alert condition at the fuel dispenser is established.

In certain aspects, the fuel cutoff device is an electronic relay positioned in line between a fuel pump of said fuel dispenser and a power supply to said fuel pump, wherein the fuel cutoff device includes a relay to prohibit supply of power from said power supply to said fuel pump.

In certain aspects, the fuel cutoff device is a valve positioned in line between a fuel pump and a fuel tank of said fuel dispenser, wherein the fuel cutoff device includes an electronically actuated valve to prohibit supply of fuel from said fuel tank to said fuel pump.

In certain aspects, the fuel dispenser is a gas pump or a fuel transfer pump coupled to a fuel storage container.

In certain aspects, the fuel monitoring system is removable coupled with said fuel dispenser.

In certain aspects, the at least one sensor includes a differential pressure sensor to monitor a differential pressure across a dispensing fuel filter at said fuel dispenser.

In certain aspects, an alert condition at the fuel dispenser is established when the differential pressure across the dispensing fuel filter deviates from a predetermined range.

In certain aspects, the portable user device signals the alert condition.

In certain aspects, the at least one sensor includes a flow meter to monitor flow of fuel through a dispensing fuel filter at said fuel dispenser, wherein an alert condition at the fuel dispenser is established when the flow through the dispensing fuel filter deviates from a predetermined range.

In certain aspects, the at least one sensor dynamically monitors cleanliness of fuel at said fuel dispenser and said measurement data reflects the cleanliness of said fuel, wherein the portable user device analyzes the measurement data and, based on the measurement data, identifies one or more dispensing fuel filters that are most suitable for the fuel.

In certain aspects, the portable user device enables an operator to purchase said one or more dispensing fuel filters via the portable user device.

In certain aspects, the at least one sensor includes a temperature sensor to monitor a temperature at said fuel dispenser, such as a temperature of the fuel or a temperature of a component of the fuel dispenser.

### DESCRIPTION OF THE DRAWINGS

These and other advantages of the present invention will be readily understood with the reference to the following specifications and attached drawings, where like reference numbers refer to like structures. The figures are not necessarily to scale, emphasis is instead placed upon illustrating the principles of the devices, systems, and methods described herein.

FIG. 1 illustrates an isometric view of an example dispending fuel filter having portions thereof removed to expose filter components within housing.

FIG. 2 illustrates a diagram of an example fuel evaluation and monitoring system coupled to plural fuel dispensers, plural fuel monitoring apparatus, and a portable user device.

FIG. 3 illustrates a diagram of an example fuel dispenser.

FIG. 4 illustrates a diagram of an example fuel monitoring apparatus.

FIGS. 5a and 5b illustrate an example fuel monitoring apparatus embodied as a stand-alone monitoring apparatus.

FIGS. 6a through 6d illustrate an example standalone anti-theft cutoff device.

FIGS. 7a and 7b illustrate example screenshots of an example operator interface as displayed to the operator on a portable user device.

### DETAILED DESCRIPTION

Preferred embodiments of the present invention will be described herein with reference to the accompanying drawings. In the following description, well-known functions or constructions are not described in detail because they could obscure the invention in unnecessary detail.

All documents mentioned herein are hereby incorporated by reference in their entirety. References to items in the singular should be understood to include items in the plural, and vice versa, unless explicitly stated otherwise or clear from the text. Grammatical conjunctions are intended to express any and all disjunctive and conjunctive combinations of conjoined clauses, sentences, words, and the like, unless otherwise stated or clear from the context. Thus, the term “or” should generally be understood to mean “and/or” and so forth.

Recitation of ranges of values herein are not intended to be limiting, referring instead individually to any and all values falling within the range, unless otherwise indicated herein, and each separate value within such a range is incorporated into the specification as if it were individually recited herein. The words “about,” “approximately,” or the like, when accompanying a numerical value, are to be construed as indicating a deviation as would be appreciated by one of ordinary skill in the art to operate satisfactorily for an intended purpose. Ranges of values and/or numeric values are provided herein as examples only, and do not constitute a limitation on the scope of the described embodiments. The use of any and all examples, or exemplary language (“e.g.,” “such as,” or the like) provided herein is merely intended to better illuminate the embodiments and does not pose a limitation on the scope of the embodiments. No language in the specification should be construed as indicating any unclaimed element as essential to the practice of the embodiments.

In the following description, it is understood that terms such as “first,” “second,” “top,” “bottom,” “side,” “front,” “back,” and the like are words of convenience and are not to be construed as limiting terms. Further, the word “exemplary” means “serving as an example, instance, or illustration.” The embodiments described herein are not limiting, but rather are exemplary only. It should be understood that the described embodiments are not necessarily to be construed as preferred or advantageous over other embodiments. Moreover, the terms “embodiments of the invention,” “embodiments,” or “invention” do not require that all embodiments of the invention include the discussed feature, advantage, or mode of operation.

The terms “communicate” and “communicating” as used herein, include both conveying data from a source to a destination and delivering data to a communications medium, system, channel, network, device, wire, cable, fiber, circuit, infrared, and/or link to be conveyed to a destination. The term “communication” as used herein means data so conveyed or delivered. The term “communications” as used herein includes one or more of a communications medium, system, channel, network, device, wire, cable, fiber, circuit, and/or link.

The terms “coupled,” “coupled to,” and “coupled with” as used herein, each mean a relationship between or among two or more devices, apparatuses, files, circuits, elements, functions, operations, processes, programs, media, components, networks, systems, subsystems, and/or means, constituting any one or more of: (i) a connection, whether direct or through one or more other devices, apparatuses, files, circuits, elements, functions, operations, processes, programs, media, components, networks, systems, subsystems, or means; (ii) a communications relationship, whether direct or through one or more other devices, apparatuses, files, circuits, elements, functions, operations, processes, programs, media, components, networks, systems, subsystems, or means; and/or (iii) a functional relationship in which the operation of any one or more devices, apparatuses, files, circuits, elements, functions, operations, processes, programs, media, components, networks, systems, subsystems, or means depends, in whole or in part, on the operation of any one or more others thereof.

The term “data” as used herein means any indicia, signals, marks, symbols, domains, symbol sets, representations, and any other physical form or forms representing information, whether permanent or temporary, whether visible, audible, acoustic, electric, magnetic, electromagnetic, or otherwise manifested. The term “data” is used to represent predetermined information in one physical form, encompassing any and all representations of corresponding information in a different physical form or forms.

The term “database” as used herein means an organized body of related data, regardless of the manner in which the data or the organized body thereof is represented. For example, the organized body of related data may be in the form of one or more of a table, map, grid, packet, datagram, frame, file, email, message, document, report, list, or in any other form.

The term “network” as used herein includes both networks and inter-networks of all kinds, including the Internet, and is not limited to any particular network or inter-network.

The term “processor” as used herein means processing devices, apparatuses, programs, circuits, components, systems, and subsystems, whether implemented in hardware, tangibly embodied software, or both, and whether or not it is programmable. The term “processor” as used herein includes, but is not limited to, one or more computing devices, hardwired circuits, signal-modifying devices and systems, devices and machines for controlling systems, central processing units, programmable devices and systems, field-programmable gate arrays, application-specific integrated circuits, systems on a chip, systems comprising discrete elements and/or circuits, state machines, virtual machines, data processors, processing facilities, and combinations of any of the foregoing.

As will be described below, a fuel evaluation and monitoring system in accordance with an aspect of the present invention may be configured to detect a condition of the dispensing fuel filter, the cleanliness of fuel, the presence of water in the fuel, and/or unauthorized use of a fuel dispenser. For example, in addition to monitoring fuel quality, the fuel evaluation and monitoring system may monitor differential pressure across a dispensing fuel filter to indicate whether the dispensing fuel filter has accumulated sufficient contaminant to warrant replacement or inspection thereof, in which case the fuel evaluation and monitoring system may suggest a suitable replacement dispensing fuel filter based on at least one sensor that dynamically monitors cleanliness of fuel at said fuel dispenser to generate measurement data that reflects the cleanliness of said fuel. The fuel evaluation and monitoring system may further provide an anti-theft feature where, in response to detection of unauthorized usage, the flow of fuel from the fuel dispenser is disabled. The anti-theft feature may be further configured to disable the fuel dispenser during predetermined time periods (e.g., after normal business hours), thereby mitigating unauthorized use of the fuel dispenser.

The disclosed fuel evaluation and monitoring system may be applied to existing fuel dispensers, such as those found at convenience stores, fuel stations, and/or fuel transfer pumps 300b used in connection with fuel storage containers. For example, a fuel evaluation and monitoring system in accordance with the present disclosure may employ one or more sensors positioned inline between the fuel tank and the fuel nozzle of a fuel dispenser sensor to dynamically monitor a parameter of said fuel dispenser or a volume of fuel passed by said fuel dispenser. As can be appreciated, the disclosed fuel evaluation and monitoring system, or components thereof, may be integrated with fuel dispensers (e.g., during manufacture) or, in certain aspects, provided in a modular, stand-alone fashion to enable after-market retrofit of existing fuel dispensers. For example, the fuel monitoring system 400, or portions thereof (e.g., sensors, valves, relays, etc.), may be housed in a single housing that does not require invasive modifications to the fuel dispenser.

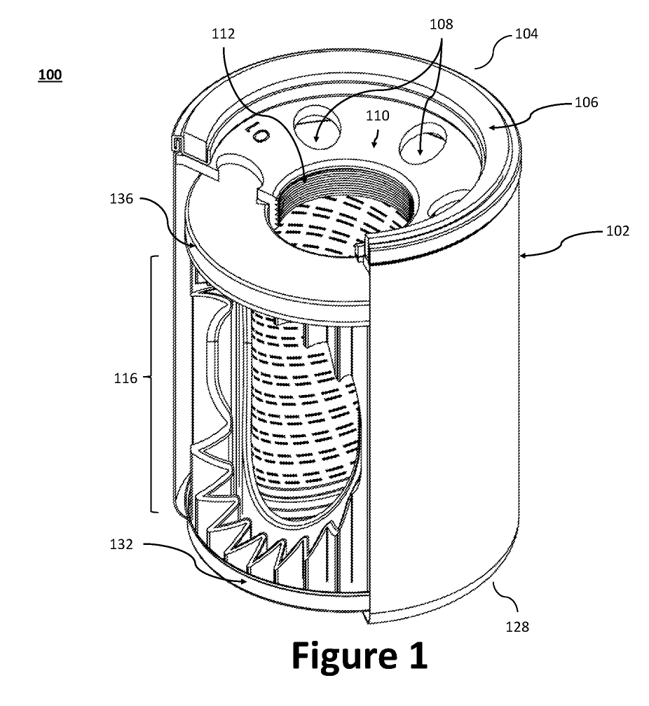

FIG. 1 illustrates an isometric view of an example dispensing fuel filter 100 having portions thereof removed to expose the filter components within filter housing 102. Dispensing fuel filters 100, such as those available from Cim-Tek® Filtration, are designed to accumulate particulate. As illustrated, the dispensing fuel filter 100 may comprise a filter housing 102 (e.g., a canister) having an open end 104 and a closed end 128. The filter housing 102 may be configured to receive a filter assembly, the filter assembly generally comprising a closed end cap 132, an open end cap 136, and a filter element 116 positioned therebetween. The filter assembly generally operates to filter particulate, and in some instances, detect water. An example water-sensing filter is disclosed by commonly owned U.S. Pat. No. 9,381,453 titled Fuel Filter, which issued on Jul. 5, 2016.

While the filter element 116, and components thereof, are illustrated as being generally cylindrical, other shapes and designs are contemplated. To secure the filter assembly within the filter housing 102, a threaded end plate 110 may be coupled to the open end 104 of the filter housing 102. The threaded end plate 110 may be coupled to the filter housing 102 using one or more fixed securing techniques, including, for example, crimping, adhesives, welding, rivets, etc., or removable securing techniques (e.g., threadedly coupled).

The threaded end plate 110 may comprise a threaded hole 112 positioned at an approximate center of a circular plane defined by the top surface of the threaded end plate 110. A plurality of holes 108 (e.g., about 2 to 10, more preferable about 2 to 8, most preferable about 6) are further arranged around the threaded hole 112. In operation, the plurality of holes 108 serve as a fuel inlet to the dispensing fuel filter 100, while the threaded hole 112 of the end plate 110 serves as a fuel outlet. Preferably, the area of the threaded hole's 112 opening is equal to, or greater than, the cumulative area of the plurality of holes 108's openings so as to ensure that the outlet can accommodate fuel flow from the inlet. The threaded hole 112 may be sized and configured to couple to a fuel delivery system, such as a fuel dispenser 300 or a stand-alone monitoring apparatus 500 coupled to a fuel dispenser 300. An external seal 106 is further provided along the top circumference of the open end 104, which allows the filter housing 102 to form a fluid tight seal with a corresponding mating component of the fuel delivery system. While the plurality of holes 108 serve as the fuel inlet to the dispensing fuel filter 100 in the illustrated example, one of skill in the art would appreciate that other configurations are possible.

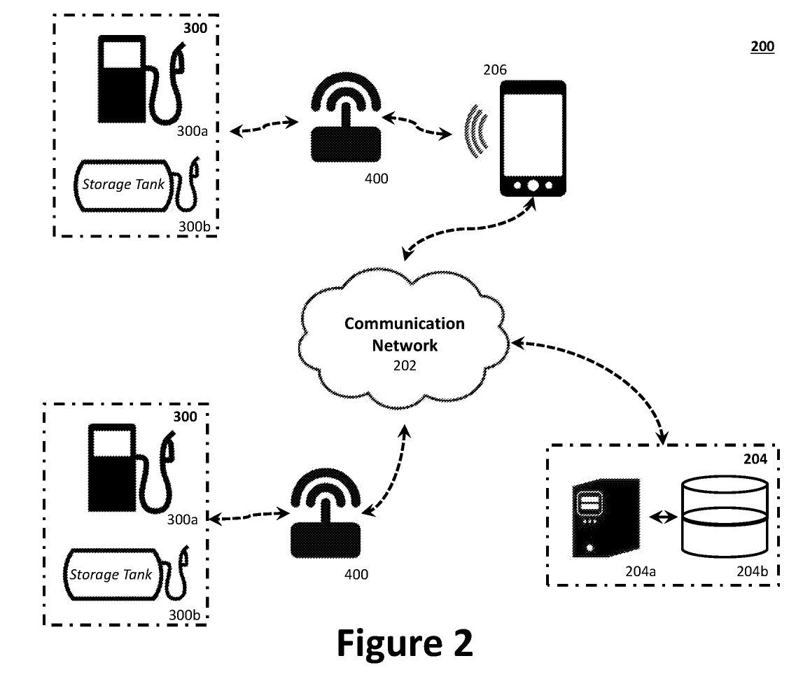

An example fuel evaluation and monitoring system 200 is illustrated in FIG. 2. The fuel evaluation and monitoring system 200 facilitates communication (e.g., directly or via communication network 202) between one or more portable user devices 206 (e.g., a portable computer, tablet, smart phone, or other device), one or more fuel monitoring systems 400, and a remote fuel evaluation and monitoring server 204. While the fuel evaluation and monitoring system 200 is generally described as using a portable user device 206, the operator may similarly access the fuel monitoring system 400 or the remote fuel evaluation and monitoring server 204 over the communication network 202 via an online operator portal (e.g., via an Internet or intranet webpage).

The fuel evaluation and monitoring system 200 is operable to collect and report fuel quality or fuel equipment status to one or more portable user devices 206 and/or a remote fuel evaluation and monitoring server 204. For example, the fuel evaluation and monitoring system 200 may detect and report, via a fuel monitoring system 400, fuel quality, contaminates, cleanliness, and/or free or emulsified water in the fuel, as well as the status of a fuel dispenser 300 or a dispensing fuel filter 100.

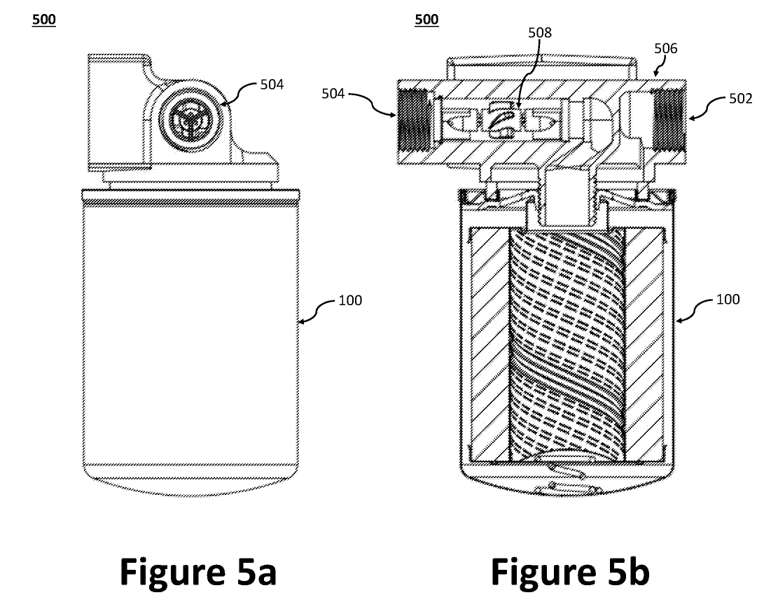

The one or more fuel monitoring systems 400 may be operably coupled with one or more fuel dispensers 300 (e.g., gas station pumps 300a, such as those found at convenience stores and fuel stations, and/or fuel transfer pumps 300b used by fuel storage containers). As will be discussed, each fuel monitoring system 400 includes one or more sensors to gather information relating to fuel quality, status of the dispensing fuel filter 100, and/or status of the fuel dispensers 300. The fuel monitoring system 400 may either be integral with fuel dispensers 300 (e.g., integrated during manufacture) or configured as an after-market to retrofit existing fuel dispensers 300. Accordingly, the fuel monitoring system 400, or portions thereof (e.g., sensors, valves, etc.), may be housed in a single housing positioned inline between the fuel dispenser's 300 fuel tank 302 and fuel nozzle 310 without requiring any invasive modifications to the fuel dispenser 300; an example of which is illustrated in FIGS. 5a and 5b.

While the communication network 202 is illustrated as a single network (e.g., the Internet), one of skill in the art would recognize that one or more communication networks may be used to facilitate communication between the various components of the fuel evaluation and monitoring system 200. Moreover, an encrypted communication channel, such as Secure Sockets Layer (“SSL”), may be employed to communicate data between, for example, the fuel dispensers 300 and remote fuel evaluation and monitoring server 204. In addition to, or lieu of, the communication network 202, each portable user device 206 may communicate directly with the fuel monitoring system 400 of the fuel dispenser 300 via point-to-point communication (e.g., without requiring an intervening network or node).

The fuel monitoring system 400 may communicate information (e.g., measurement data, which may be pre-processed or unprocessed) to the portable user device 206 or a base station (e.g., a router or communication relay) via one or more communication protocols. The one or more communication protocols include, for example, long and short range wireless communication, such as Bluetooth (e.g., short-wavelength, UHF radio waves in the ISM band from 2.4 to 2.485 GHz), Wi-Fi (e.g., IEEE 802.11), near field communication (NFC), ZigBee (e.g., IEEE 802.15.4), radio frequency (RF) (e.g., 900 MHz), infrared, and/or cellular networks. The portable user device 206 may directly communicate wirelessly with the fuel monitoring system 400 via Bluetooth, ZigBee, RF, NFC, infrared, etc. For example, measurement data from one or more sensors (e.g., sensor measurement data, such as raw signal values, signals, or data values) may be sent to the portable user device 206 as unprocessed measurement data for processing (whether performed at the portable user device 206 or the remote fuel evaluation and monitoring server 204). Alternatively, the measurement data from one or more sensors may be processed by the fuel monitoring system 400 to generate an alert, which may be sent to the portable user device 206.

The remote fuel evaluation and monitoring server 204 generally comprises a processor (e.g., computer 204a) configured to perform one or more algorithms/protocols and a non-transitory data storage device 204b. Analysis/processing of sensor measurement data may be performed locally (e.g., at the fuel dispenser 300 or the fuel monitoring system 400). Alternatively, the sensor measurement data from one or more sensors may be reported to the one or more portable user devices 206 and/or the remote fuel evaluation and monitoring server 204 as unprocessed measurement data (e.g., as raw sensor measurement data from one or more sensors) for further analysis, in which case the unprocessed measurement data may be remotely processed by the portable user device 206 or by the remote fuel evaluation and monitoring server 204. Accordingly, the fuel monitoring system 400 may capture and communicate unprocessed measurement data to the portable user device 206 or the fuel evaluation and monitoring server 204 without the need to pre-process the measurement data, thereby reducing the equipment needed at the fuel monitoring system 400.

In this case, the portable user device 206 or the fuel evaluation and monitoring server 204 processes the unprocessed measurement data to yield operator readable results (e.g., data values, charts, tables, suggestions, etc.), thereby obviating the need for local processing of data. Mitigating the need for locally processing equipment at the point of use (i.e., only requiring the measurement and signal generation devices) greatly reduces the operator's barrier to entry by mitigating the costs associated with the integration of the fuel monitoring system 400. For example, the unprocessed measured data may be communicated to the portable user device 206 and ultimately stored to the fuel evaluation and monitoring server 204, where the operator can access and manipulate the measurement data through an application installed on the portable user device 206. Such a fuel monitoring system 400 is particularly advantageous to relatively inexperienced operators who lack experience with differential pressure sensors 426, switches, gauges, and the like, while also providing analytic results in a fraction of the time.

Whether the information is analyzed locally, by the remote fuel evaluation and monitoring server 204, the portable user device 206, or elsewhere, the results may be used to alert the operator of a problem via the portable user device 206 and/or to provide analytic information. The analytic information would not only be useful for historical trend analysis and filter life monitoring, but also for troubleshooting or correlating filter life issues to historical events, which may be provided as a dynamic data feed or manually by the operator (e.g., upon request). Example historical events can include heavy rain, a new delivery of fuel, etc. The analytic information may also be used to suggest, and purchase, an appropriate replacement dispensing fuel filter 100 based on the fuel currently being used.

The remote fuel evaluation and monitoring server 204 may be further configured to selectively regulate or disable individual fuel dispensers 300, thereby selectively disabling flow fuel from a given fuel dispenser 300. For example, as will be discussed below, the remote fuel evaluation and monitoring server 204 may disable the flow of fuel from a fuel tank 302 that has been identified as containing contaminated fuel by outputting a control signal to the fuel dispenser 300 (via dispenser processor 314) or the fuel monitoring system 400 (via processor 402) to slow or discontinue fuel flow. In another example, the remote fuel evaluation and monitoring server 204 may disable the flow of fuel if unauthorized use (e.g., theft) is detected. For example, an electronic relay may disconnect power to the fuel pump 304. In another example, an electronically actuated valve (e.g., an electronically actuated flow cutoff solenoid valve) may be positioned inline that, when actuated, prohibits fuel flow to the fuel nozzle 310. Such cutoff valves may be integral with the fuel dispenser 300, or provided inline as an aftermarket product that does not require communication with the fuel dispenser 300.

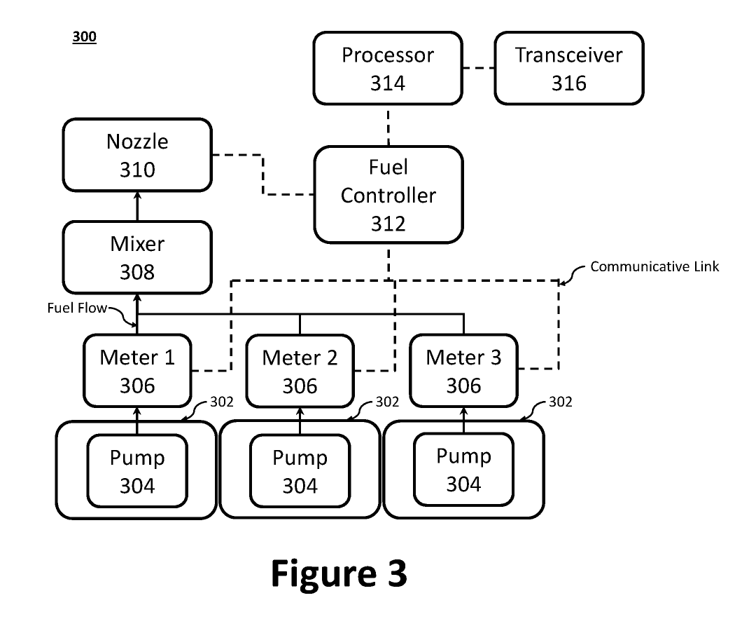

FIG. 3 provides illustrates a simplified block diagram of an example fuel dispenser 300. As mentioned above, the fuel dispensers 300 may be, for example, a gas station pump 300a and/or a fuel transfer pump 300b coupled to a fuel storage container. The fuel dispenser 300 may comprise a head component containing a mechanical device or embedded computer, which are configured to, inter alia, control the action of the pump 304, drive the fuel dispenser's 300 display(s), and, in certain aspects, communicate with a sales system (e.g., a point of sale system), whether co-located or remote. The fuel dispenser 300 may further comprise a pumping component having, for example, an electric motor, pumping unit, meters, pulsers, and/or valves, which work together to physically pump and control the fuel flow from a fuel tank 302 to a fuel nozzle 310. As illustrated, a fuel dispenser 300 generally comprises one or more fuel-fuel pumps 304 (e.g., one within an above ground fuel storage container or one positioned within an underground fuel tank 302), one or more meters 306, a mixing manifold 308, a fuel nozzle 310, a fuel controller 312, a dispenser processor 314, and a transceiver 316. The dispenser processor 314 may be a Central Processing Unit (CPU)) that is operatively coupled to a Read-Only Memory (ROM) for receiving one or more instruction sets, to a Random Access Memory (RAM) having a plurality of buffers for temporarily storing and retrieving information, and to an internal data storage device.

The various components may be coupled with one another via one more hoses, or other conduit, capable of carrying fuel. Further, the fuel controller 312 and/or dispenser processor 314 may be configured to control or monitor the various components using wired or wireless communication techniques or devices (e.g., cable, wireless transceivers, etc.).

Each of the one or more fuel-fuel pumps 304 may be operatively coupled with a meter 306. Each of the meters 306 being coupled with the mixing manifold 308, this is coupled to the fuel nozzle 310 via a hose. The fuel controller 312 is communicatively coupled with, for example, the meters 306, and the dispenser processor 314. The dispenser processor 314 may be configured to communicate information to or from the fuel dispenser 300 via the transceiver 316. For example, if the dispenser processor 314 generates an alert pertaining to an operational function of the fuel dispenser 300 or a parameter of the fuel, the alert may be conveyed to a remote device, such as a point of sale device or a portable user device 206. The fuel dispenser 300 may likewise receive information from, for example, the portable user device 206 or the remote fuel evaluation and monitoring server 204, via the transceiver 316. For example, a remote fuel evaluation and monitoring server 204 may provide instructions to the dispenser processor via the transceiver 316. While it is contemplated that the transceiver 316 would provide wireless communication, wired communication techniques may also be employed.

The fuel-fuel pump 304 may be, for example, a turbine pump. Fuel in the fuel tank 302 may be passed through a strainer or filter, which removes any solid particles, prior to entering the fuel pump 304. Any quantities of trapped air and/or fuel vapor may also be removed from the fuel through an air separator chamber. The fuel, free of air and vapor, may pass through a control valve that permits fuel to flow only in the direction of the meter 306. That is, fuel does not pass back to the pump. The control valve may be mechanical or a solenoid-controlled pilot valve.

The one or more meters 306 may employ piston meters and be of positive-displacement. For instance, a piston moving through a cylinder filled with liquid will displace a quantity of liquid, which will be determined by the bore of the cylinder and the stroke of the piston. The pistons operate may operate in a horizontal plane or in a vertical plane and convert to from a reciprocating action to a rotary shaft output, which can drive either a sensor or a mechanical gearbox. The fuel from the one or more meters 306 is mixed via the mixing manifold 308, which conveys the fuel to the fuel nozzle 310 via a hose (or other conduit).

The fuel controller 312 is communicatively coupled with the one or more meters 306 such that signals indicative of the liquid flow rate can be transmitted from the meters 306 to the fuel controller 312. Preferably, the one or more meters 306 are pulsers, such as are commonly used in gasoline dispensers. In operation, the pulsers emit a pulse for, for example, every 1/1000th of a gallon of gasoline passed by the meter 306. Thus, as the fuel is being pumped, a pulse train is delivered on the respective lines, with the pulse train frequencies corresponding to the liquid flow rate. The liquid pumps may, of course, be located elsewhere within the fuel dispenser 300 or fuel tank 302, and may have the metering devices integral with them.

The fuel controller 312 is also operatively coupled with the dispenser processor 314 that controls the overall operation of the fuel dispenser 300. For example, the dispenser processor 314 can transmits signals to the fuel controller 312 indicating that pumping is desired or to disable pumping, when the fuel controller 312 has ascertained that pumping should be disabled (e.g., based on fuel quality, meeting dispense threshold, etc.).

The fuel dispenser 300 may further include a transceiver 316 that is configured to convey data directly to a portable user device 206 and/or over a communication network 202 to a remote location (e.g., remote fuel evaluation and monitoring server 204) or a portable user device 206. The transceiver 316 may be wired or wireless and may convey, for example, the status of the fuel dispenser 300, the dispensing fuel filter 100, or parameters pertaining to the fuel quality (e.g., fuel cleanliness, water content, etc.).

In certain aspects, the remote fuel evaluation and monitoring server 204 or portable user device 206 may be operable to control the authorization of fueling transactions and other operations of the fuel dispenser 300. For example, the fuel dispenser 300 may be remotely activated or deactivated via the fuel controller 312 based on a signal from the remote fuel evaluation and monitoring server 204 or portable user device 206. The remote fuel evaluation and monitoring server 204 or portable user device 206 may be in communication with each point of sale, which may be integral with the fuel dispenser 300. Additional information regarding the general structure and operation of a fuel dispenser 300 may be gleaned from U.S. Pat. No. 7,948,376 to Jonathan E. DeLine, entitled “Fuel dispenser,” and U.S. Patent Pub No. 2014/0071073 to Rodger K. Williams, entitled “Fuel dispenser 300 Having Electrophoretic Grade Select Assembly.”

While not shown, the fuel dispenser 300 may further comprise a vapor recovery subsystem having a vapor return line from the fuel nozzle 310 and a vapor impulsion device to induce vapor to flow through the vapor return line at a vapor flow rate comparable to the liquid flow rate through the fuel delivery line during a fueling operation. An example vapor recovery subsystem is illustrated by U.S. Pat. No. 5,345,979 to Mark B. Tucker, entitled “High Efficiency Vapor Recovery Fuel Dispensing.” While the fuel dispenser 300 is illustrated as having multiple fuel-fuel pumps 304 and multiple fuel tanks 302, one of skill in the art would appreciate that a fuel evaluation and monitoring system in accordance with the subject disclosure may be applied to systems having a single fuel-fuel pump 304 and/or fuel tank 302. For example, the transceiver 316 may be omitted if there is no need to convey data to or from the fuel dispenser 300. Moreover, elements of the fuel dispenser 300 may be omitted in simplified variants, such as fuel transfer pumps 300b coupled to fuel storage containers. As can be appreciated, in embodiments where the fuel dispenser 300 is a fuel transfer pump 300b coupled to a fuel storage tank; the fuel dispenser 300 may be simplified by omitting unnecessary components.

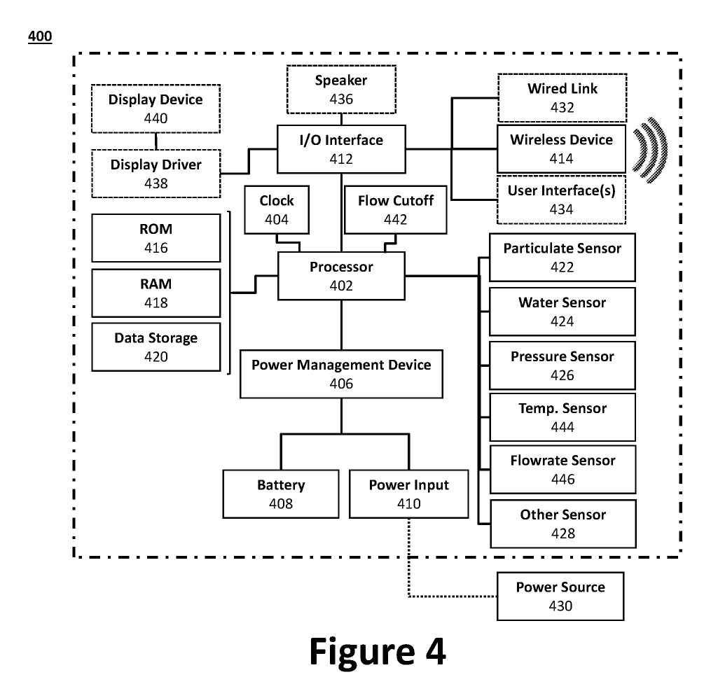

FIG. 4 illustrates a block diagram of an example fuel monitoring system 400 suitable for use in connection with the fuel evaluation and monitoring system 200 of FIG. 2. The fuel monitoring system 400 may be integrated with a fuel dispenser 300 or, in other aspects, the fuel evaluation and monitoring system may be a standalone device configured to filter and monitor fuel received from, or to be dispensed from, a fuel dispenser 300 (e.g., prior to deliver to an engine or other fuel tank, such as a vehicular fuel tank). An example stand-alone monitoring apparatus 500 is illustrated in FIGS. 5a and 5b.

As illustrated in FIG. 4, the fuel monitoring system 400 includes a processor 402 (e.g., a CPU) that is operatively coupled to a ROM 416 for receiving one or more instruction sets, to a RAM 418 having a plurality of buffers for temporarily storing and retrieving information, and to an optional internal data storage device 420 (e.g., a non-volatile data storage device, such as flash memory, including removable memory cards). In certain aspects, the fuel monitoring system 400 may employ a removable memory device (e.g., a flash memory card) to store data collected over a period of time. The processor 402 may be communicatively coupled with the dispenser processor 314 and/or the fuel controller 312 of a given fuel dispenser 300, which may analyze the signal to determine whether a pumping operation should be regulated (e.g., by stopping the fuel pump 304). A clock 404 is also coupled to the processor 402 for providing clock or timing signals or pulses thereto. Those skilled in the art will understand that the fuel monitoring system 400 may include one or more bus structures for interconnecting the various components.

The fuel monitoring system 400 may further include an input/output interface 412 that interfaces the processor 402 with one or more peripheral and/or communicative devices, such as a wireless device 414, which may be a wireless transmitter or transceiver. In certain aspects, the processor 402 may be coupled with one or more optional devices, such as operator interface(s) 434, a wired link 432, and/or a speaker 436, which may be used to signal an alert or other status information pertaining to the fuel filter or flow. In certain situations, the fuel monitoring system 400 may include security features, such as a fuel cutoff device 442, which may include an electronic relay or an electronically actuated valve. Fuel flow may be regulated or restricted by controlling the operation of the fuel pump 304. For example, an electronic relay may be positioned in line between a fuel pump 304 of said fuel dispenser 300 and a power supply to said fuel pump 304, in which case the electronic relay is operable to prohibit supply of power from said power supply to said fuel pump 304, thereby prohibiting flow of fuel. In another example, an electronically actuated valve may be positioned in line between a fuel pump 304 (or other device) and the fuel tank 302 of said fuel dispenser 300, in which case the electronically actuated valve prohibits supply of fuel from said fuel tank to said fuel pump through selective opening and closing of the electronically actuated valve.

In other words, the fuel cutoff device 442 is operable to prohibit flow from the fuel tank 302 to the fuel nozzle 310. For example, the processor 402 may toggle a fuel cutoff device 442 to disable use of the fuel dispenser 300 during certain time periods or for particular operators (e.g., requiring a password, pin, etc.). The fuel cutoff device 442 may be integral with the fuel dispenser 300, or positioned inline as an aftermarket solution to prohibit fuel flow.

The wireless device 414 may be configured to manage communication and/or transmission of signals or data between the processor 402 and another device (e.g., a portable user device 206 via communication network 302 or directly with a portable user device 206) by way of a wireless transceiver. The wireless device 414 may be a wireless transceiver configured to communicate via one or more wireless standards such as Bluetooth, NFC, Wi-Fi, Zigbee, RF, etc. For example, wireless connectivity (e.g., RF 900 MHz or Wi-Fi) may be integrated with the fuel monitoring system 400 to provide remote monitoring and control the fuel monitoring system 400 via one or more portable user devices 206. In certain aspects, an internal cellular modem may be implemented that utilizes standards-based wireless technologies, such as 2G, 3G, 4G, code division multiple access (CDMA), and Global System for Mobile Communications (GSM), to provide wireless data communication over worldwide cellular networks.

The fuel monitoring system 400 may further comprise a plurality of sensors to dynamically (e.g., in real time or near real time) collect, generate, and/or communicate measured data in real-time, at predetermine times or intervals (whether regular or irregular intervals), or upon a trigger (e.g., request by the operator). For example, one or more sensors may be provided inline between the fuel tank 302 and the fuel nozzle 310 in intimate contact with the fuel, such as at the fuel nozzle 310, mixing manifold 308, hose, or even the one or more meters 306. Depending on the sensor type, the sensors of the fuel monitoring system 400 may be positioned inline between the tank 302 and the fuel nozzle 310 to detect one or more fuel quality parameters, or one each side of the dispensing fuel filter 100 to determine a differential pressure across the fuel filter. Positioning a sensor at the fuel nozzle 310, for instance, enables the fuel monitoring system 400 to dynamically monitor the fuel as it is being dispensed, which provides the most accurate indication of what is being dispensed.

As illustrated, the fuel filter 100 may be coupled directly to the stand-alone monitoring apparatus 500 to enable differential pressure measurements across the fuel filter without requiring additional sensors. The sensor's measured parameter (e.g., particle count, water concentration, conductivity, etc.) may be communicated from the one or more sensors to a remote processor as measurement data in the form of unprocessed measurement data. The remote processor may analyze the unprocessed measurement data to determine a parameter of the fuel. For example, the measured parameter may be communicated from the fuel monitoring system 400 to a remote fuel evaluation and monitoring server 204 or a portable user device 206 for analysis. The sensors may be used to detect one or more monitored parameters. For example, the fuel monitoring system 400 may include a particulate sensor 422, a water sensor 424, a differential pressure sensor 426, a temperature sensor 444, a flowrate sensor 446, and other sensors 428.

When the measured parameter exceeds a predetermined threshold, an alert signal may be generated to signal an alert condition (e.g., when a shut off threshold is reached) for the given fuel. That is, an alarm (or other alert) may be provided by the portable user device 206 and/or the fuel dispenser 300 may be disabled. For example, a look of table may be used by one or more of the fuel dispenser 300, the fuel monitoring system 400, the remote fuel evaluation and monitoring server 204, and/or the portable user device 206 to ensure that a measured parameter is within a normal operating range.

Particulate Sensor 422.

In order to detect the fuel cleanliness, one or more particulate sensors 422 may employ particle-counting techniques, which may be used to determine a cleanliness code (e.g., ISO 4406 fuel cleanliness). As noted above, cleanliness is particularly applicable to diesel fuels, but also applies to other fuels, such as gasoline. Thus, the particulate sensor 422 may be a particle counter configured to analyze the fuel and determine the ISO 4406 fuel cleanliness at the fuel dispenser 300 (or other fuel transfer point). The nature of particle counting may be based upon, inter alia, either light scattering, light obscuration, or direct imaging. The particulate sensor 422 further includes the various electrical and mechanical components useful to determine particle counts and to generate a base signal (4-20 mA, voltage, etc.), which need not be processed locally by the fuel dispenser 300 or the fuel monitoring system 400.

Water Sensor 424.

The water sensor 424 is configured to detect water content (e.g., in gasoline ethanol blends, where phase separation is a risk). For example, one or more water sensors 424 may be positioned along the fuel line between the fuel-pumping unit 304 and the fuel nozzle 310 and configured to perform conductivity measurements of the fuel adjacent the sensor, which may be used to detect the presence and amount of water. The conductivity of fuel (e.g., gasoline) varies depending on the water concentration. For example, ethanol and gasoline mixtures with some water content will provide a characteristic electrical signal that differs from ethanol and gasoline mixtures with different water content. The bulk electrical conductivity may be measured using an impedance sensor. For example, conductivity of gasoline is typically about 25 picosiemens per meter (pS/m), while the conductivity of no. 2 diesel is typically about 5 pS/m. As the concentration of water in the fuel increases, the conductivity of the fuel solution increases. Thus, the water sensor 424 may employ an impedance sensor to determine when the conductivity of the fuel deviated from a predetermined range.

Differential Pressure Sensor 426.

A differential pressure sensor 426 may be used to determine whether a dispensing fuel filter 100 has reached the end of its service life. As contaminants are accumulated in the dispensing fuel filter 100, or as the flow is restricted by a water sensing material, flow resistance through the dispensing fuel filter 100 increases. That is, the flow resistance of fuel increases from the inlet (e.g., plurality of holes 108) where fuel enters the dispensing fuel filter 100 to the outlet (e.g., threaded hole 112) where the fuel exits the dispensing fuel filter 100. Once the dispensing fuel filter 100 has accumulated sufficient contaminant or other blockage to cause the flow resistance to increase to achieve a predetermined value (e.g., a terminal flow resistance), the dispensing fuel filter 100 is deemed to be at the end of its useful life. The recommended predetermined value is typically established or determined when a given dispensing fuel filter 100 is designed. The flow resistance can be measured through, for example, differential pressure, which refers to the difference between the system pressure upstream of the dispensing fuel filter's 100 filtering material and the system pressure downstream of the dispensing fuel filter's 100 filtering material. In a typical fuel transfer or fuel dispensing applications, for example, dispensing fuel filters 100 are designed to be removed from service at approximately 20 to 30 psid, more preferably about 25 psid. As is appreciated by those of skill in the art, psid refers to a measurement of the pressure differential between two pressures.

A differential pressure sensor 426 determines the difference in pressure between two points in a system (e.g., upstream and downstream of the dispensing fuel filter 100) using one pressure sensor positioned adjacent the inlet side and one pressure sensor positioned adjacent the outlet side of the filter 100. If a measured differential pressure deviates from the normal operating range, an alert may be generated to signal the alert condition. For example, where the fuel monitoring system 400 is operable to process the measurement data, the fuel monitoring system 400 may send an alert to the operator's portable user device 206 when the differential pressure reaches the dispensing fuel filter's 100 terminal pressure rating to signal to the operator that it is time to replace the filter 100. As noted above, processing of measurement data may be performed remotely, however. In certain aspects, the differential pressure sensor 426 may employ a magnetic movement that allows the simultaneous sensing of both pressures while completely isolating the differential pressure gauge function from the pressure chamber without requiring mechanical seals. In instances where high and low limit control is desired, two sets of differential pressure sensors 426 may be installed. In certain aspects, the differential pressure sensor 426 may be processor controlled and equipped with unique Hall Effect sensors to convert a traditional differential pressure gauge's normal magnetic movements into electric signals.

In addition to dispensing fuel filter 100 status monitoring, the cleanliness of the fuel delivered over a predetermine period of time may be determined using a differential pressure sensor 426. As can be appreciated, it is advantageous to determine, or predict, the volume of fuel that a particular fuel filter can handle prior to requiring service or replacement of the fuel filter. While a manufacturer can typically predict the volume of fuel that a particular fuel filter can process prior to requiring service or replacement under ideal circumstances, the actual volume, however, depends on a number factors. For example, incoming fuel quality (e.g., particulate level and water concentration) influences the volume that can be processed before achieving a terminal flow resistance or other predetermined value. That is, contaminated fuel typically expedites flow restriction, which is a byproduct of the filter clogging process, in addition to general aging and degradation of the filter material.

Since the flow resistance is affected by the fuel quality, the flow resistance measured over time may be used as an indication of the fuel quality supplied to that dispensing fuel filter 100. Thus, given the flow resistance value, which may be measured in real time, the fuel quality may be determined in real time. For example, a look up table may be used to correlate measured differential pressure with cleanliness levels for various fuel and/or fluid types. Accordingly, the portable user device 206 and/or remote fuel evaluation and monitoring server 204 may be configured to provide a corresponding fuel cleanliness level based at least in part on differential pressure. A large change in differential pressure coupled with specific filter features may be used to identify a significant fuel issue, such as catastrophic ingression of water, fuel that is heavily laden with particulate contamination, etc.

Temperature Sensor 444.

In mechanical systems, an objective is typically to transfer energy from one part of the system to another, where it can be transferred into motion. A normal part of this process is the loss of energy to heat, but an efficient system will keep that energy loss to a minimum. An increase in temperature indicated inefficiencies in the system. Temperature also influences fuel and characteristics thereof; therefore, a temperature sensor 444 may be used as a diagnostic tool. Areas of interest are those of water tolerance of given fuels, cloud and gel point for diesel fuels, fluid viscosity etc. With input specifications from fuel employed, an operator may use temperature in early detection scenarios. Particularly when coupled with alternate features herein to signal when in range for water in neat fuels to separate into free or emulsified water, or when phase separation is likely to occur for those fuels blended with Ethanol or the like. A temperature sensor 444 may be included in the housing to measure any notable changes in temperature of the working fluid, such as fuel. This could be done via thermocouple, an electronic method of translating voltage to temperature. The dynamic temperature reading could be accessible by the operator via the portable user device 206.

Flowrate Sensor 446.

The flowrate sensor 446 may be, for example, a turbine flow meter, a paddle wheel flow meter, nutating disk flow meter, etc. A turbine flow meter translates the mechanical rotation of a turbine into a readable flowrate such as GPM. The turbine is housed in the path of the fluid stream, which will rotate the angled blades of the turbine and set the turbine in motion. The rotational speed of the turbine is proportional to the fluid velocity. Metal inserts may be embedded into the blades of the turbine, which are then picked up by a magnetic sensor, creating an electrical pulse signal. The frequency of this pulse signal is proportional to the turbine rotation speed, and therefore the fluid flowrate. The signal is then translated and displayed as a readable unit of flow such as GPM. Similar to the turbine flow meter, a paddle wheel flow meter operates by translating mechanical flow into electrical signals. The difference comes in the mechanism used. While a turbine takes a reading from axial flow, a paddle wheel takes a reading from radial flow. With regard to the nutating disk flow meter, a disk is mounted eccentrically within a housing, with the bottom and top of the disk in contact with the housing chamber. An opening in the disk separates the inlet and outlet of the chamber. As fluid flows through the chamber, it forces the disk to nutate about the vertical axis. Each full nutation of the disk correlates to a fixed volume, which allows for accurate measurement of fluid flowrate through the chamber. This style of flow meter is best utilized for low-flow situations, but well within the required flowrates for fuel dispensers 300.

A power management device 406 may be used to manage power needed to operate the fuel monitoring system 400 (and components thereof). That is, power may be drawn from a power input 410 and/or an internal battery 408. The fuel monitoring system 400 may further comprise alternate power sources, such as a solar panel to enable maintaining and charging of the internal battery 408.

The fuel monitoring system 400 may further comprise one or more optional components. In certain aspects, for example, an optional wired link 432 may be provided to manage communication and/or transmission of signals or data between the processor 402 and another device via, for example, a data port capable of being wiredly coupled with a data port positioned outside the fuel monitoring system 400 housing (e.g., the fuel dispenser 300). As illustrated, the processor 402 may be optionally operatively coupled to a display device 440 via a display driver 438. The display device 440 may comprise one or more light emitting diodes (LEDs), or a liquid crystal display (LCD) screen to display one or more menus, icons, or text. An optional operator interface 434 may be used to enable the operator to adjust the settings of the fuel monitoring system 400. Example operator interface(s) 434 devices may include, for example, physical buttons, physical switches, a digitizer (whether a touch pad, or transparent layer overlaying the display device 440), and other input devices.

Direct communication between the portable user device 206 and the fuel dispensers 300 or fuel monitoring system 400 may obviate the need for an interface and supporting hardware on the fuel dispenser 300 or fuel monitoring system 400 for interpreting the signals from the sensor and/or displaying them in an operator understandable format. Thus, the initial unit cost is minimized when unprocessed measurement data is communicate to a portable user device 206 or remote fuel evaluation and monitoring server 204 to interpret and display the information via portable user device 206.

The various components of a fuel monitoring system 400 may be housed in a single housing as a modular, stand-alone monitoring apparatus 500 to increase ease of use and as an aftermarket solution. FIGS. 5a and 5b illustrate an example stand-alone monitoring apparatus 500 with an integral flowrate sensor 446 suitable for use in connection with fuel evaluation and monitoring system 200. The stand-alone monitoring apparatus 500 enables economic and efficient fuel management by integrating a flowrate sensor 446 into a piece of equipment that can be readily integrated into a fuel dispenser 300. The stand-alone monitoring apparatus 500 includes a housing 506 that defines a fluid inlet 502 and a fluid outlet 504. The housing 506 includes a flowrate sensor 446 adjacent the fluid outlet 504. The stand-alone monitoring apparatus' 500 housing 506 couples to a filter 100 (e.g., via threaded hole 112). Fuel from the fluid inlet 502 travels through the filter 100 and exits the housing 506 via the fluid outlet 504. As the fluid flows across the flowrate sensor 446, the flowrate sensor 446 generates a reading based on the velocity of the fuel. The stand-alone monitoring apparatus 500 may include one or more of a particulate sensor 422, a water sensor 424, a differential pressure sensor 426, a temperature sensor 444, a flowrate sensor 446, and other sensors 428. The stand-alone monitoring apparatus 500 may be configured to couple with a dispensing fuel filter 100, thereby offering a direct replacement for existing dispensing fuel filter 100 couplings. The stand-alone monitoring apparatus 500 may be installed inline to allow the operator to monitor, for example, the flowrate of the fuel being dispensed.

The stand-alone monitoring apparatus 500 that may detect and analyze differential pressure and volume, without requiring hard-wired control circuit connections, thereby enabling the operator to quickly and efficiently interpret and document one or more fuel filter service life conditions. A stand-alone monitoring apparatus 500 in accordance with an aspect of the subject disclose is easy to deploy, easy to initialize, easy to maintain, and would allow for the documentation and manipulation of historical data without manual data entry, evaluation, complex and sophisticated controls, and data logging devices. That is, the stand-alone monitoring apparatus 500 may facilitate documentation and manipulation of historical data without requiring manual data entry and evaluation or complex controls and data logging devices. For example, where cost is a factor, the flow monitoring apparatus may be configured without one or more of a manual interface, a readout (e.g., display or other indicator, such as LEDs), and external hard-wired connections. As with the fuel monitoring system 400, the operator may dynamically export a live data feed to the portable user devices 206 through a mobile application. As the data is recorded, the stand-alone monitoring apparatus' 500 internal processor may compile the collected data and send this information wirelessly to the portable user device 206 as unprocessed measurement data or, in the alternative, as an operator readable format for display at the portable user device 206. For example, the export may be transferred to one or more portable user devices 206 via Bluetooth, which may then be passed to a remote fuel evaluation and monitoring server 204.

Antitheft System.

Fueling systems located in remote locations are often the target of fuel theft and unauthorized dispensing, which results in issues relating to fuel reconciliation efforts, etc. Existing theft deterrent devices inhibit the ability to physically remove the fuel nozzle 310, but can be easily defeated through use of common hand tools. Accordingly, a need exists for a cost effective security solution that restricts unauthorized fuel dispensing without requiring manual onsite intervention. The fuel evaluation and monitoring system 200 may be configured to restrict or otherwise control access to the fuel dispensers 300 by remotely disabling the supply of power to a fuel dispenser 300 (e.g., via the fuel controller 312 or at the fuel pump 304) or via a fuel cutoff device 442 that physically restricts the flow of fuel. For example, the fuel evaluation and monitoring system 200 may be configured to restrict access only to those individual operators who provide predetermined credentials (e.g., a password, pin, biometric information, etc.). The fuel evaluation and monitoring system 200 allows management to remotely enable or disable use of the fuel dispensers 300, which may be on a scheduled basis. In order to make the logging of fuel usage a mandatory process, the fuel evaluation and monitoring system 200 may be further configured to disable the fuel dispensers 300 unless all required inputs are logged and/or required credentials are provided. Alternatively, fuel conditions or cleanliness could be used as trigger for disabling the pump should this remote relay be used in conjunction with integrated filter adaptor described herein.

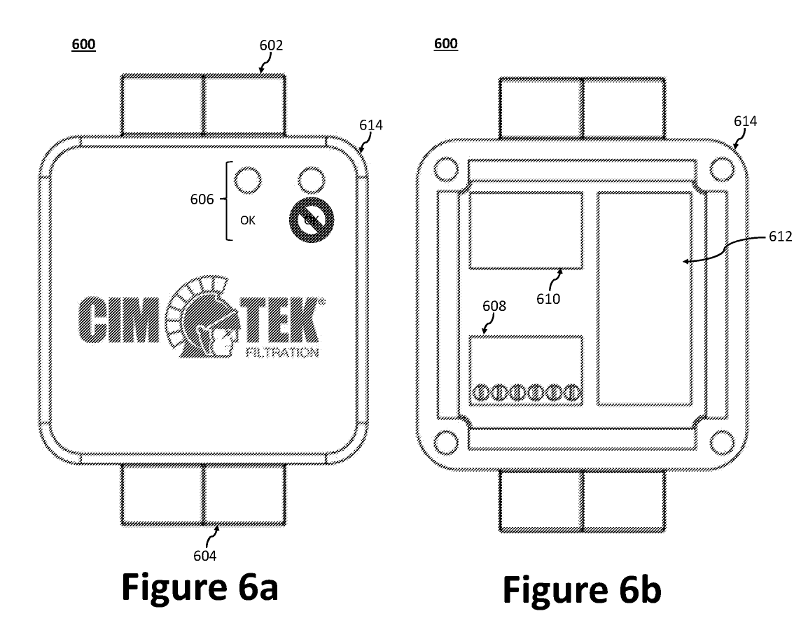

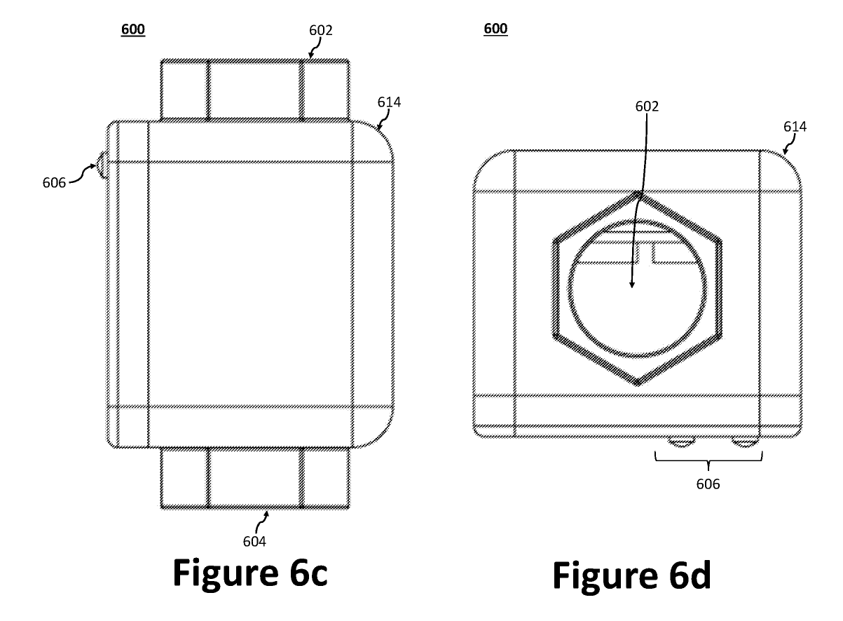

In certain aspects, a standalone inline anti-theft cutoff device 600 may be installed fluidly inline between a fuel tank and a fuel nozzle. FIGS. 6a through 6d illustrate, respectively, front, rear, side, and top plan views of an example anti-theft cutoff device 600 for installation inline between a fuel tank and a fuel nozzle. The anti-theft cutoff device 600 generally comprises a housing 614, a fuel inlet 602, a fuel outlet 604, a plurality of display devices 440 (e.g., indicators 606), terminal connections 608, a communication device 610 (e.g., wired link 432, wireless device 414, and/or operator interface 434), and one or more electric relay modules 612 to actuate a fuel cutoff device 442. The indicators 606 can indicate via an LCD or LED display, for example, whether the anti-theft cutoff device 600 is activated (prohibiting flow) or deactivated (permitting use).

The electronics may be housed in materials suited for use in open environments where exposure to various weather conditions, dust debris, etc. are accommodated. Suitable materials include both metallic and non-metallic materials, which may be coated or otherwise treated for outdoor use, depending on specific challenge conditions. The housing 614 may be sized to accommodate the communication devices (e.g., a wireless receiver), one or more electrical relays for desired pump amperage ranges/voltage conditions, and terminal connections 608 for use in various wiring configurations. Various seal configurations are available for incoming wiring including, flexible cord grips, NPT for rigid conduit, etc. In some instances, the indicators 606 may include an LCD, color indicating LEDs, or alternate indicators to provide current system status of the anti-theft cutoff device 600. The anti-theft cutoff device 600 may operate in one of multiple ways. One method contemplated is for the anti-theft cutoff device 600 to include an electric relay module 612 to disconnect the fuel pump 304 from its power supply, thereby disabling it.

The anti-theft cutoff device 600 may be field installable inline of source for electrically powered fuel transfer pumps 300b through retrofit or new installation systems. Installation may be required between the power source to the fuel pump 304 and the location of the fuel pump 304. In another example, the electric relay module 612 may be configured to actuate an electronically actuated valve that prohibits the flow of fuel through the electronically actuated valve. The anti-theft cutoff device 600 could be configurable through installation instruction for various common power sources (120 vac, 220 vac, 12 vdc, etc.), and interrupt thereof. The anti-theft cutoff device 600 provides the ability to allow or disallow the operation of the fuel dispenser 300 based on an acceptable set of input criteria having been met prior to attempt of dispensing.

The anti-theft cutoff device 600 may be controlled remotely to provide on/off control of power source via remote fuel evaluation and monitoring server 204 or a portable user device 206. For example, the anti-theft cutoff device 600 may provide wireless connectivity to operator's portable user device 206 via Bluetooth connection or some other wireless communication platform to enable additional flexibility in utilization. Operation could be performed with connectivity capability with or without the integrated connected filter adaptor. Operation with allows additional disabling features such as when filter conditions and or fuel quality are suspect. For example, as described in connection with fuel monitoring system 400, the anti-theft cutoff device 600 may prohibit the operation of the fuel dispenser 300 (e.g., by cutting the power supply to the fuel pump 304 or prohibiting flow of fuel via a fuel cutoff device 442) when a measured parameter deviated from a normal operating range (e.g., indicating an alert condition), thereby alerting the operator or site manager of suspect fuel conditions. If the fuel dispenser 300 is disabled based on a measured parameter, the operator may be alerted via the portable user device 206 that additional steps are necessary to reset the system for operation (e.g., an alert condition, such as an indication of a deviating measured parameter, such as the fuel's water content, particle count, etc.), which may require a filter change, tank cleaning, fuel treatment, etc. before returning to desired service.

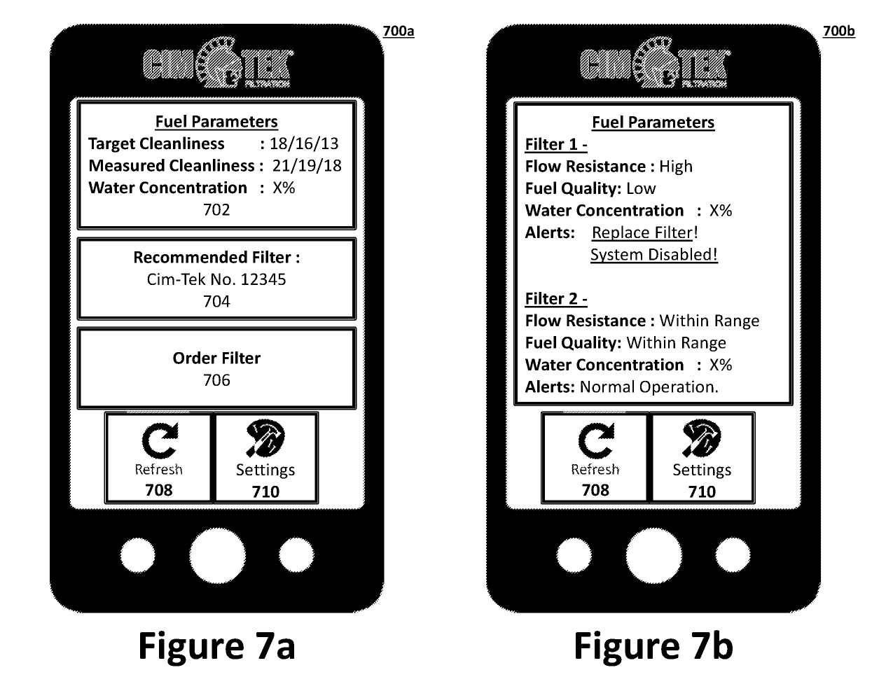

Portable user device Interface. FIGS. 7a and 7b illustrate an example screenshots 700a, 700b of an operator interface as displayed to the operator on a portable user device 206. The portable user device 206 may receive the various sensors' readings and, based upon the sensor's reading, display one or more monitored parameters in near real time (e.g., as the operator is dispensing fuel into equipment). The particle count, for example, may be displayed in accordance with ISO 4406 cleanliness code. The various data may be sent to a remote server (e.g., remote fuel evaluation and monitoring server 204) for storage and further evaluation, thereby facilitating historical trending that may be used to provide analysis on individual fueling or fuel transfer points.

The portable user device 206 will display the measurement data as one or more parameters, including, for example, (1) flowrate readings; (2) differential pressure readings; (3) estimated remaining filter life; (4) volume of fluid dispensed; (5) system power status (enabled/disabled), etc. an operator may also input information at the time of dispensing via the portable user device 206 to facilitate the collection of operator-defined data including, but not limited to: employee ID; task ID; vehicle ID; job number; contract number, etc. The operator-defined data may be used in connection with authenticating the user as part of the anti-theft system.

The operator-defined data, along with the measurement data compiled from the flow meter, enables an operator-friendly means for the consumer to monitor their fuel usage, which allows bulk fuel operators to identify discrepancies. Upon detection of unclean fuel and/or water, the fuel evaluation and monitoring system 200 may alert the operator or operator while, in certain aspects, automatically prohibiting the flow of fuel from the tank (e.g., by disabling the fuel pump 304 or activating a fuel cutoff device 442). For example, an alert may be sent from the fuel evaluation and monitoring system (which may be integral with a fuel dispenser 300) to a portable user device, such communication may be either direct, or through a network. The fuel evaluation and monitoring system may provide additional features, such as an anti-theft system.

As illustrated in FIG. 7a, the portable user device 206 may display fuel parameters 702, a recommended filter 704, an option to order a filter 706 (e.g., the recommended filter), a refresh option 708, and a settings option 710, which may enable the operator to, inter alia, change his or her fuel or filter preferences or setting. Using the settings option 310, the operator may, for example, enter the fuel type, a target cleanliness code, the filter type installed on the fuel dispenser 300, vehicle, etc. The portable user device 206 may further display, or sound, one or more alerts indicating, for example, that the water concentration has reached a predetermined concentration. The portable user device 206 may be configured to receive the various sensor data from the fuel dispensers 300 (e.g., via a particle counter, water sensor, etc.) and, based on the measured data, calculate and display the contamination level of the fuel being pumped (e.g., a calculated cleanliness code). For example, as illustrated, the target cleanliness code may be 18/16/13, while the calculated cleanliness code is 21/19/18, thereby indicating that the fuel or too dirty. While the contamination level is illustrated in terms of a cleanliness code, other data representations may be displayed or provided including, for example, the measured data, an alert icon, etc.).

Using the portable user device 206, the operator may input the filter type currently installed, and based on the target cleanliness code and calculated cleanliness code, the portable user device 206 may provide a recommendation as to which filter would be preferred for the fuel, possibly with secondary and tertiary options. For example, one or more one sensors may dynamically monitor cleanliness of fuel at the fuel dispenser to generate measurement data reflecting the cleanliness of the fuel, in which case the portable user device analyzes the measurement data and, based on the measurement data, identifies one or more dispensing fuel filters that are most suitable for the fuel using, for example, a look up table.

The portable user device 206 may be further configured to link, or otherwise direct, the operator to a merchant that sells the suggested filter. For example, the operator may select the filter and purchase it via the portable user device 206 using the Internet. The fuel evaluation and monitoring system 200 obviates the time consuming process of taking samples, sending them for analysis, and seeking the needed filter by enabling the used to perform all of these steps in real time while the equipment is done being refueled. In certain embodiments, the certain of the teachings disclosed herein may be integrated with a vending machine (e.g., at an auto supply store, gas station, etc.), whereby a fuel sample may be inserted for analysis, along with other data parameters, and a suggested filter may be dispensed. The vending machine may also provide a payment system, whether integral or communicatively coupled with another device (e.g., a portable user device).

With reference to FIG. 7b, the portable user device 206 may display, for each filter dispensing fuel filter 100 or fuel dispensers 300 within the fuel evaluation and monitoring system 200, various monitored parameters, such as flow resistance, fuel quality, water concentration, and/or one or more alerts. As noted above, when a monitored parameter deviates from a predetermined operation rage, the fuel evaluation and monitoring system 200 may generate an alert for display at the portable user device 206. For example, as illustrated in FIG. 7a, flow resistance is high, thereby indicating that Filter 1 requires replacement and, consequently, that the fuel dispenser 300 associated with Filter 1 may be disabled when a shut off threshold (or other predetermined threshold) is reached.

The fuel evaluation and monitoring system 200 may employ a multiple tiered alert arrangement. For example, in addition to a shut off threshold, one or more warning threshold levels may be provided where the fuel is still acceptable for use, but not ideal (e.g., the water concentration is approaching an unacceptable level). The warning thresholds may be set by one or more of the operators, including the operator (e.g., the proprietor of the fuel station), the consumer (e.g., the purchaser of the fuel), etc. The fuel monitoring system 400 enables alerts that indicating that an event occurred outside of typical filter life, and that remediation efforts may be necessary. This may include system and tank cleaning efforts, filter change, contacting fuel supplier for large-scale issues, or the like.

The above-cited patents and patent publications are hereby incorporated by reference in their entirety. Although various embodiments have been described with reference to a particular arrangement of parts, features, and the like, these are not intended to exhaust all possible arrangements or features, and indeed many other embodiments, modifications, and variations will be ascertainable to those of skill in the art. Further, while the forgoing has been described with regard to fuel dispensers, one of skill in the art would recognize that the techniques taught herein might be employed with other applications where water detection within a fluid is desired. Thus, it is to be understood that the invention may therefore be practiced otherwise than as specifically described above.

What is claimed is:

1. A fuel monitoring system for use with a fuel dispenser, the fuel monitoring system comprising:
at least one sensor to dynamically monitor a parameter of said fuel dispenser or a volume of fuel passed by said fuel dispenser;
a processor operably coupled with said at least one sensor, the processor being configured to receive measurement data from said at least one sensor that represents a monitored parameter of said volume of fuel; and
a wireless transceiver operably coupled with said processor that is configured to wirelessly communicate said measurement data from said fuel monitoring system to a portable user device.

2. The fuel monitoring system of claim 1, wherein the measurement data is wirelessly communicated to said portable user device as unprocessed measurement data.

3. The fuel monitoring system of claim 2, wherein the unprocessed measurement data is processed by said portable user device to determine whether an alert condition at the fuel dispenser is established.

4. The fuel monitoring system of claim 1, wherein the measurement data is wirelessly communicated to said portable user device using Bluetooth.

5. The fuel monitoring system of claim 1, wherein the measurement data is wirelessly communicated to said portable user device using Wi-Fi.

6. The fuel monitoring system of claim 5, wherein the measurement data is communicated to said portable user device via the Internet.

7. The fuel monitoring system of claim 1, further comprising a fuel cutoff device to disable flow of fuel from the fuel dispenser.

8. The fuel monitoring system of claim 1, wherein the fuel cutoff device is configured to disable flow of fuel from the fuel dispenser when, based on said measurement data, an alert condition at the fuel dispenser is established.

9. The fuel monitoring system of claim 7, wherein the fuel cutoff device is an electronic relay positioned in line between a fuel pump of said fuel dispenser and a power supply to said fuel pump, wherein the fuel cutoff device includes a relay to prohibit supply of power from said power supply to said fuel pump.

10. The fuel monitoring system of claim 7, wherein the fuel cutoff device is a valve positioned in line between a fuel pump and a fuel tank of said fuel dispenser, wherein the fuel cutoff device includes an electronically actuated valve to prohibit supply of fuel from said fuel tank to said fuel pump.

11. The fuel monitoring system of claim 1, wherein the fuel dispenser is a gas pump.

12. The fuel monitoring system of claim 1, wherein the fuel dispenser is a fuel transfer pump coupled to a fuel storage container.

13. The fuel monitoring system of claim 1, wherein the fuel monitoring system is removable coupled with said fuel dispenser.

14. The fuel monitoring system of claim 1, wherein the at least one sensor includes a differential pressure sensor to monitor a differential pressure across a dispensing fuel filter at said fuel dispenser.

15. The fuel monitoring system of claim 14, wherein an alert condition at the fuel dispenser is established when the differential pressure across the dispensing fuel filter deviates from a predetermined range.

16. The fuel monitoring system of claim 15, wherein the portable user device signals the alert condition.

17. The fuel monitoring system of claim 16, further comprising a fuel cutoff device to disable flow of fuel from the fuel dispenser in response to the alert condition.

18. The fuel monitoring system of claim 1, wherein the at least one sensor includes a flow meter to monitor flow of fuel through a dispensing fuel filter at said fuel dispenser, wherein an alert condition at the fuel dispenser is established when the flow through the dispensing fuel filter deviates from a predetermined range.

19. The fuel monitoring system of claim 1, wherein the at least one sensor dynamically monitors cleanliness of fuel at said fuel dispenser and said measurement data reflects the cleanliness of said fuel, wherein the portable user device analyzes the measurement data and, based on the measurement data, identifies one or more dispensing fuel filters that are most suitable for the fuel.

20. The fuel monitoring system of claim 19, wherein the portable user device enables an operator to purchase said one or more dispensing fuel filters via the portable user device.

21. The fuel monitoring system of claim 1, wherein the at least one sensor includes a temperature sensor to monitor a temperature at said fuel dispenser.
